(19) United States
(12) Patent Application Publication
(10) Pub. No.: US 2024/0127533 A1
(43) Pub. Date: Apr. 18, 2024

(54) INFERRING DEVICE, MODEL GENERATION METHOD, AND INFERRING METHOD

(71) Applicants: Preferred Networks, Inc., Tokyo (JP); ENEOS Corporation, Tokyo (JP)

(72) Inventors: So TAKAMOTO, Tokyo (JP); Wenwen LI, Tokyo (JP); Yusuke ASANO, Tokyo (JP); Takafumi ISHII, Tokyo (JP)

(73) Assignees: Preferred Networks, Inc., Tokyo (JP); ENEOS Corporation, Tokyo (JP)

(21) Appl. No.: 18/534,176

(22) Filed: Dec. 8, 2023

Related U.S. Application Data
(63) Continuation of application No. PCT/JP2022/023500, filed on Jun. 10, 2022.

(30) Foreign Application Priority Data
Jun. 11, 2021 (JP) 2021-098316

(51) Int. Cl. G06T 17/00; G06V 10/82; G06V 20/69

(52) U.S. Cl. CPC G06T 17/00; G06V 10/82; G06V 20/695

(57) ABSTRACT

An inferring device includes one or more memories and one or more processors. The one or more processors are configured to obtain three-dimensional structures of a plurality of molecules; and input the three-dimensional structures of the plurality of molecules into a neural network model and infer one or more physical properties of the plurality of molecules.

### CROSS REFERENCE TO THE RELATED APPLICATIONS

This application is continuation application of International Application No. JP2022/023500, filed on Jun. 10, 2022, which claims priority to Japanese Patent Application No. 2021-098316, filed on Jun. 11, 2021, the entire contents of which are incorporated herein by reference.

### FIELD

This disclosure relates to an inferring device, a model generation method, and an inferring method.

### BACKGROUND

In the field of novel material exploration, the field of drug discovery, and the like, producing, in silico, a chemical compound that can be used as many materials and predicting the physical properties of this compound is widely done. For example, in a field called chemoinformatics, a method to obtain an explanatory variable from a molecular structure to predict physical properties is used.

Chemoinformatics uses methods to (1) extract a characteristic structure such as a specific bond, functional group, or the like from a molecular structure to use this as an explanatory variable, (2) treat a molecule as a graph and employ a machine learning method using the graph, and (3) replace a graph by a character string and employ a method of natural language processing. In executing the prediction, all of these methods treat the molecule as a stand-alone entity without imparting information on the ambient environment of the molecule. However, a problem with these simulation methods is that it is not possible to consider what arrangement or the like a chemical compound can actually take in a substance.

There is another method called Neural Network Potential (NNP) that pays attention to the three-dimensional structure of a molecule to obtain energy. There may be a possibility to infer physical properties using this NNP, but in principle, it is not possible to calculate general physical properties only from the three-dimensional structure.

### BRIEF DESCRIPTION OF THE DRAWINGS

FIG. 1 is a block diagram illustrating an example of an inferring device according to an embodiment.

FIG. 2 is a flowchart illustrating a process of the inferring device according to the embodiment.

FIG. 3 is a block diagram illustrating an example of a model generating device according to an embodiment.

FIG. 4 is a flowchart illustrating a process of the model generating device according to the embodiment.

FIG. 5 is a diagram illustrating one example of an implementation of an information processing system/device according to an embodiment.

### DETAILED DESCRIPTION

According to one embodiment, an inferring device includes one or more memories and one or more processors. The one or more processors are configured to obtain three-dimensional structures of a plurality of molecules; and input the three-dimensional structures of the plurality of molecules into a neural network model and infer one or more physical properties of the plurality of molecules.

Embodiments of the present invention will be hereinafter described with reference to the drawings. The drawings and the description of the embodiments are presented by way of example and are not intended to limit the present invention. This disclosure describes, as one embodiment, generating a machine learning model that predicts physical properties of a material using the result of Molecular Dynamics (MD) simulation as an input variable, and executing the prediction using this machine learning model. Note that in this disclosure, the term “molecule” may include the case of an atom by itself.

### Inferring Device

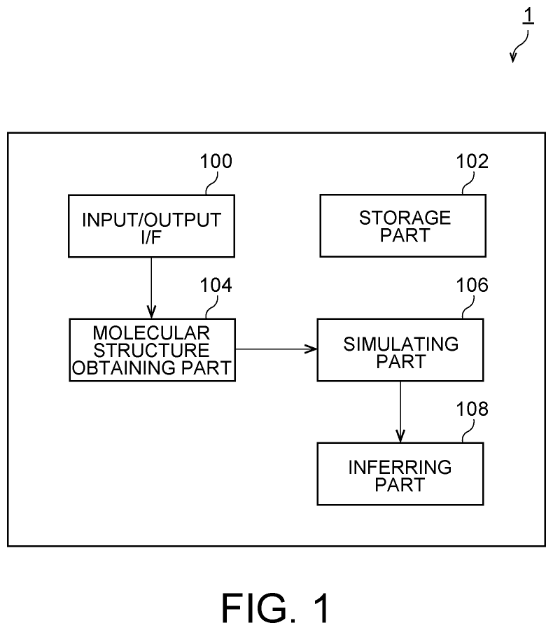

FIG. 1 is a block diagram illustrating an example of an inferring device according to an embodiment. The inferring device 1 may include an input/output interface (hereinafter, referred to as an input/output I/F 100), a storage part 102, a molecular structure obtaining part 104, a simulating part 106, and an inferring part 108.

The input/output I/F 100 may be an interface for executing the input/output of data and so on to/from the inferring device 1. In the drawing, not all of the transmissions/receipts of data through the input/output I/F 100 are illustrated, and the input/output of data may be executed through the input/output I/F 100 from/to an appropriate place at an appropriate time. For example, from an external database, the input/output I/F 100 obtains information necessary for the inference. Further, a user may input data through the input/output I/F 100.

The storage part 102 may store information necessary for the operation of the inferring device 1 and a target and a result of its processing. When necessary, the constituent elements of the inferring device 1 may access the storage part 102 to read/write data.

The molecular structure obtaining part 104 may convert input data to data regarding a molecular structure. The molecular structure obtaining part 104 obtains, for example, a molecular structure of a target material and a molecular structure of a substance forming an environment based on information such as a chemical formula. For example, the molecular structure obtaining part 104 obtains information regarding the molecular structure in a data format conforming to the input to the simulating part 106. Note that in the case where information obtained through the input/output I/F 100 or information stored in the storage part 102 is the data regarding the molecular structure, the molecular structure obtaining part 104 is not an essential structure of the inferring device 1.

The simulating part 106 may obtain a three-dimensional structure of a molecule from the data regarding the molecular structure by executing MD simulation. For example, in the case where a plurality of molecules are present in the environment, the simulating part 106 may obtain three-dimensional structures of the plurality of molecules by executing the simulation. By the simulating part 106 converting the molecule to the three-dimensional structure, it is possible to obtain information regarding a greater variety of physical properties as compared with information that can be obtained only from the information of the molecule.

The inferring part 108 may infer the physical property from the three-dimensional structure. This inference may be executed using a neural network model trained by a later-described training device (model generating device). This neural network model may be a model that outputs one or more physical properties when the three-dimensional structure of one kind of molecule or a plurality of molecules is input thereto. For accepting the input of the three-dimensional structure, this neural network model may be a model trained using a model of NNP.

NNP is a neural network trained to reproduce an interatomic interaction obtained using quantum chemical calculation. In response to the input of the coordinates of each atom, NNP infers a physical property (physical property value) to output it. This inference calculation requires a far less calculation amount than quantum chemical calculation and thus achieves simulation of a large system for which real-time inference using MD simulation or the like is difficult. The simulating part 106 may obtain the coordinates of atoms included in one kind of molecule or a plurality of molecules through simulation to output them so that the coordinates will be the input to this model. As a result, by using the model trained using the NNP model, the inferring device 1 is able to infer various physical properties to output them.

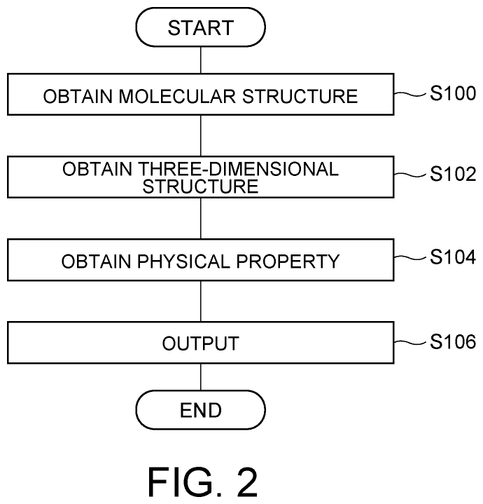

FIG. 2 is a flowchart illustrating a process of the inferring device 1 according to this embodiment.

The inferring device 1 may obtain, from an external part, data regarding a molecular structure of a material or the like whose physical property is to be obtained (S100). For example, this data may be obtained through the input/output I/F 100 or may be based on information stored in the storage part 102. The data to be obtained may be, besides the data of the material or the like, data regarding the periphery where the material or the like is present or regarding its ambient environment. For example, in the case where the inferring device 1 is used for drug discovery, information on a protein or the like to which the material bonds may be obtained as the information regarding the environment. In addition to the information on the protein, information regarding the ambient environment of the protein may be obtained together. Further, in a field other than drug discovery, the environmental information may be, for example, information on a catalyst or the like and its molecular structure may be obtained. Furthermore, a molecular structure of the periphery of a target material or its ambient environment may be obtained regardless of whether the environmental information is information on a protein or a catalyst. As previously described, if necessary, the molecular structure obtaining part 104 may execute analysis, conversion, or the like of the information input through the input/output I/F 100 to obtain the molecular structure.

Next, the simulating part 106 may obtain the three-dimensional position of atoms forming the molecule based on the obtained molecular structure (S102). As described above, the simulating part 106 uses, for example, the MD simulation method to obtain the three-dimensional arrangement of the atoms forming the molecule based on the molecular structure. According to the method used, in S100, the molecular structure obtaining part 104 may appropriately execute the conversion or the like of the format of the data to be obtained. The MD simulation method is not limited and any of the various methods is usable.

Next, the inferring part 108 may input the three-dimensional structure of the molecule (three-dimensional arrangement of the atoms) into the neural network model to obtain the physical property (S104). According to the target physical property, the neural network model has been appropriately trained. That is, depending on the target physical property, the neural network model may be changed. As another example, based on a neural network model optimized to be capable of obtaining a plurality of physical properties, the inferring part 108 may obtain a plurality of target physical properties. Further, additional information such as temperature or pressure besides the three-dimensional structure is also inputtable to this neural network model. In this case, the inferring device 1 may obtain the additional information as input information in advance and may input this additional information as well to the neural network.

Then, the inferring device 1 may output the physical property inferred by the inferring part 108 and end the process (S106). Of course, it may continue to execute the process of obtaining a plurality of physical properties or may subsequently execute a process of obtaining a physical property of a different material. In this manner, the process from S100 to S106 may be repeatedly executed a necessary number of times while the condition and so on are varied. Here, “output” may mean to output through the input/output I/F 100 to an external part or may mean to store the inferred data in the storage part 102.

As described above, according to this embodiment, by generating, through simulation, the arrangement that the molecule can actually take in the material, it is possible to predict various physical properties of the molecule with this arrangement taken into consideration. By thus generating the arrangement that can be taken in the material and inputting this to the neural network, it is possible to improve the accuracy of the physical property prediction.

Many of the physical properties attracting attention in the industry tend to be strongly governed by an intermolecular interaction. For example, physical properties regarding dynamics such as viscosity and diffusion coefficient and physical properties regarding thermal properties such as specific heat, heat conductivity, phase transformation point, and catalyst yield are strongly influenced by interactions between molecules in a material and between molecules in the material and its environment. Then, the intermolecular interaction depends on the three-dimensional arrangement of the molecules in a space. Therefore, that the information on the arrangement of molecules can be treated as the input leads to an improvement in the accuracy of the inference of physical properties.

Further, since the three-dimensional structure instead of the molecule is input to the neural network model used in the inferring part 108, the prediction of the physical properties of a mixture of a plurality of materials can be executed within the same framework. For example, even if a new material such as an additive is to be added, new training does not necessarily have to be executed and it is possible to use the same model.

### Model Generating Device

Next, a model generating device that generates the neural network model used in the inferring part 108 in the above-described inferring device 1 will be described.

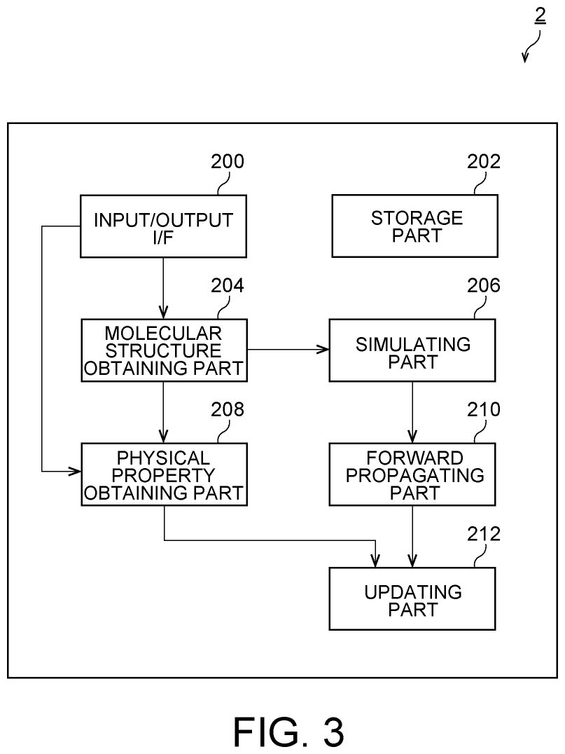

FIG. 3 is a diagram illustrating an example of the model generating device according to the embodiment. The model generating device 2 may include an input/output I/F 200, a storage part 202, a molecular structure obtaining part 204, a simulating part 206, a physical property obtaining part 208, a forward propagating part 210, and an updating part 212.

The input/output part I/F 200, the storage part 202, the molecular structure obtaining part 204, and the simulating part 206 may have the same functions as those of the above-described inferring device 1. Hereinafter, it is assumed that these constituent elements execute the same operations as those of the inferring device 1 unless otherwise mentioned.

The physical property obtaining part 208 may obtain a physical property from input data. This physical property may be a physical property that is to be output from a model to be trained. The model generating device 2 may optimize the model, using the physical property obtained by the physical property obtaining part 208 as teacher data. Similarly to the molecular structure obtaining part 204, if the physical property itself can be obtained through the input/output I/F 200 or can be obtained from the storage part 202, the physical property obtaining part 208 is not an essential structure of the model generating device 2. Here, the physical property used as the teacher data may be a generally known physical property or may be a physical property actually obtained through experiments.

The forward propagating part 210 may input the input data to an input layer of the training target model to forward propagate it. The forward propagating part 210 may input the input data to the model to execute the forward propagation processing, thereby obtaining an output from the model.

This model is, for example, a neural network model based on NNP. Specifically, when the three-dimensional structure including the three-dimensional arrangement of a plurality of atoms is input as an explanation variable to the input layer, a predetermined objective variable may be output from an output layer. As an example, the objective variable is a physical property to be inferred in the inferring device 1. Further, as in the description of the aforesaid inferring device 1, this model may be a model to which additional information such as temperature or pressure besides the three-dimensional structure is inputtable.

The updating part 212 may compare the objective variable output by the forward propagating part 210 and the teacher data obtained by the physical property obtaining part 208 and optimize a parameter of the model. For example, the updating part 212 updates the parameter of the model by executing error backpropagation. Any of the various mechanical learning methods is appropriately applicable to this updating processing.

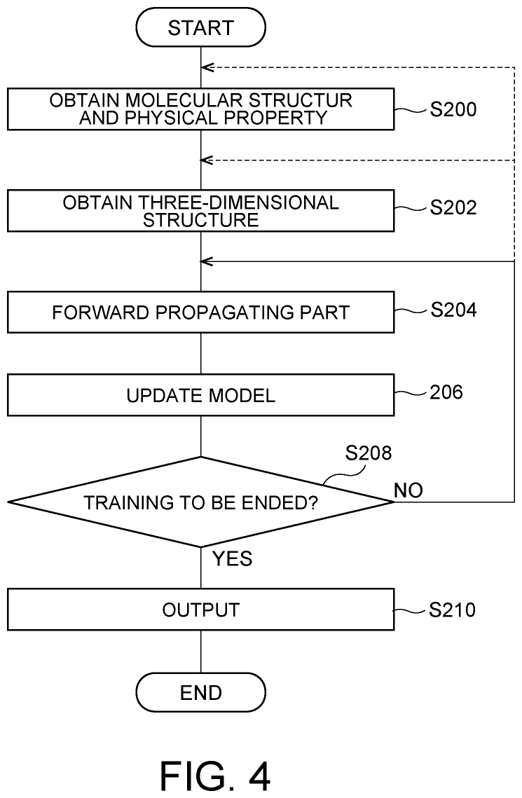

FIG. 4 is a flowchart illustrating a process of the model generating device 2.

The model generating device 2 may obtain data necessary for model generation from an external part (S200). For example, this data may be obtained through the input/output I/F 100 or may be based on information stored in the storage part 102. The data to be obtained may be, besides data of a material or the like, data regarding the periphery where the material or the like is present or its ambient environment. For example, in the case where a model to be used for drug discovery is to be generated, information on a protein or the like to which the material bonds or information regarding its ambient environment may be obtained together. The data necessary for the model generation may be data regarding a molecular structure corresponding to the input data of the inferring device 1 and data regarding a physical property that is to be output from the model. If necessary, the molecular structure obtaining part 204 may obtain the data regarding the molecular structure based on the input data or the like, and the physical property obtaining part 208 may obtain the data regarding the physical property based on the input data or the like.

Next, the simulating part 206 may obtain the three-dimensional structure of atoms forming the molecule based on the obtained molecular structure (S202). The simulating part 206 uses, for example, an MD simulation method to obtain the three-dimensional arrangement of the atoms forming the molecule from the molecular structure. According to the method used, in S200, the molecular structure obtaining part 204 may appropriately execute the conversion or the like of a format of the data to be obtained. The MD simulation method is not limited and any of the various methods is usable.

Next, the forward propagating part 210 may forward propagate the obtained data of the three-dimensional structure to the model (S204). By executing the forward propagation processing, the physical property regarding the three-dimensional structure may be obtained based on the current model. In the case where additional information is inputtable to the model, the additional information may be input together.

Next, the updating part 212 may compare an output of the model obtained by the forward propagating part 210 and the physical property obtained in S200 and update the parameter of the model (S206). The updating of the model may be executed using any of the various error backpropagation methods or may be executed using another appropriate method.

After updating the parameter, the updating part 212 may determine whether to end the training or not (S208). In the case where the training is to be ended (S208: YES), the updating part 212 may output necessary data and the model generating device 2 may end the process. In the case where the training is to be continued (S208: NO), the model generating device 2 repeats the process from, for example, S204. The process may be repeated from S200 or S202 instead of S204. In these cases, with appropriate data being obtained, the process may be repeated. Whether to end the process may be determined, for example, based on a condition such as that an evaluation value becomes smaller/larger than a predetermined value or that processing of a predetermined number of epochs is finished. Besides, based on an appropriate training end condition, the process may be ended.

The model generating device 2 may execute the processing of at least one of S200 to S210 by parallel processing. The parallel processing may be executed using an accelerator such as a multi-core processor or a many-core processor. The accelerator may be one usable through a server or the like provided on a cloud.

As described above, according to this embodiment, it is possible to generate a model capable of obtaining the physical property value in response to the input of the three-dimensional structure. Since the three-dimensional structure obtained through the MD simulation can be the input data, it is possible to increase the data amount of the training data. For example, even in a task where a molecule is an input and a corresponding physical property is to be predicted, executing the MD simulation many times regarding the same molecule makes it possible to greatly increase the amount of a training data set. This nature can contribute to an improvement in the generalization performance of input/output data.

Making use of the model of NNP as the model used in the inferring device 1 or as the model generated in the model generating device 2 can bring about the aforesaid effects. It is usually difficult to convert a three-dimensional structure to a molecular graph and thus it is difficult to adapt it to a neural network where a method applicable to a graph, such as graph convolution, is used for a molecular graph. Using the NNP model makes it possible to generate a model that obtains a physical property in which a target substance and an ambient environment of the substance are taken into consideration, without modifying the data.

Note that, in the above-described model generating device 2, the physical property is data obtained through experiments or the like, for instance. By way of a modification example, this physical property as the teacher data may be inferred through MD simulation. In the case where the physical property is obtained through the MD simulation, the model generating device 2 is also capable of generating a model using a plurality of three-dimensional structures after executing simulation once. Therefore, in the case where the physical property is to be obtained after such a model is generated, it is possible to obtain the physical property in a short time using a trained model without executing the MD simulation. In the model generating device 2 thus using the result of the MD simulation as the training data, the simulating part 206 instead of the physical property obtaining part 208 in FIG. 3 may obtain the physical property. The result is usable a plurality of times in iteration from the forward propagation to the updating in the training.

Further, in the model generating device 2, the simulating part 206 is not an essential structure. Another adoptable configuration is to calculate a plurality of three-dimensional structures through MD simulation in advance, store them in an external or internal memory or the like, and execute the training using the stored data. This also applies to the calculation of the physical property through the MD simulation.

The inference and the model generation in the above-described embodiments are applicable to, for example, a solid such as a crystal and an amorphous substance, a small molecule, a polymer, and so on. As the physical property, a thermophysical property such as heat conductivity, melting point, boiling point, diffusion coefficient, and so on can be predicted.

Further, it has been described that the inferring device 1 and the model generating device 2 are separate devices, but using the model generated as the model generating device 2, the same device as the model generating device 2 may be used as the inferring device 1.

Some or all of each device (the information processing device or an information processing device which is belonging to the information processing system 1) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

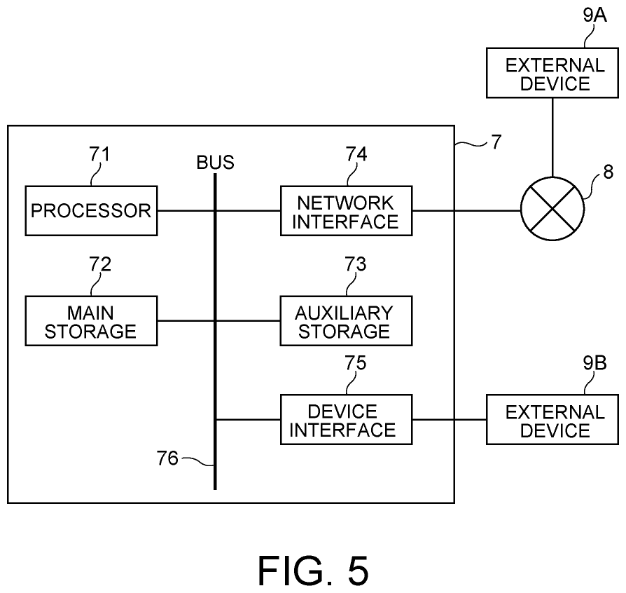

FIG. 5 is a block diagram illustrating an example of a hardware configuration of each device (the inferring device 1 or the model generating device 2) in the above embodiments. As an example, each device may be implemented as a computer 7 provided with a processor 71, a main storage 72 (hereinafter a main storage device 72), an auxiliary storage 73 (hereinafter an auxiliary storage device 73), a network interface 74, and a device interface 75, which are connected via a bus 76.

The computer 7 of FIG. 5 is provided with each component one by one but may be provided with a plurality of the same components. Although one computer 7 is illustrated in FIG. 5, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inferring device 1 or the model generating device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inferring device 1 or the model generating device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuity, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inferring device 1 or the model generating device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inferring device 1 or the model generating device 2) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the inferring device 1 or the model generating device 2) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inferring device 1 or the model generating device 2) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,”“based on data,”“according to data,”“in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
one or more memories; and
one or more processors configured to:
obtain three-dimensional structures of a plurality of molecules; and
input the three-dimensional structures of the plurality of molecules into a neural network model and infer one or more physical properties of the plurality of molecules.

2. The inferring device according to claim 1,
wherein the one or more processors are configured to obtain the three-dimensional structures of the plurality of molecules through molecular dynamics simulation.

3. The inferring device according to claim 1,
wherein the three-dimensional structures of the plurality of molecules are information regarding arrangement of atoms forming the plurality of molecules.

4. The inferring device according to claim 1,
wherein the three-dimensional structures of the plurality of molecules further include information regarding arrangement of atoms forming ambient environments of the plurality of molecules, and
wherein the one or more processors are configured to:
obtain three-dimensional structures of the environments and the three-dimensional structures of the plurality of molecules; and
input the three-dimensional structures of the plurality of molecules and the three-dimensional structures of the environments into the neural network model and infer the one or more physical properties.

5. The inferring device according to claim 1,
wherein additional information in addition to the three-dimensional structures of the plurality of molecules is inputtable to the neural network model.

6. An inferring device comprising:
one or more memories; and
one or more processors configured to:
obtain a three-dimensional structure of one molecule through molecular dynamics simulation; and
input the three-dimensional structure of the one molecule into a neural network model and infer one or more physical properties of the one molecule.

7. The inferring device according to claim 6,
wherein the three-dimensional structure of the one molecule is information regarding arrangement of atoms forming the one molecule.

8. The inferring device according to claim 6,
wherein the three-dimensional structure of the one molecule further includes information regarding arrangement of atoms forming an ambient environment of the one molecule, and
wherein the one or more processors are configured to:
obtain a three-dimensional structure of the ambient environment as well as obtain the three-dimensional structure of the one molecule; and
input the three-dimensional structure of the one molecule and the three-dimensional structure of the ambient environment into the neural network model and infer the one or more physical properties.

9. The inferring device according to claim 6,
wherein additional information in addition to the three-dimensional structure of the one molecule is inputtable to the neural network model.

10. A model generation method comprising:
by one or more processors,
obtaining three-dimensional structures of a plurality of molecules;
inputting the three-dimensional structures of the plurality of molecules into a neural network model to perform forward propagation processing, and outputting one or more physical properties of the plurality of molecules; and
updating the neural network model based on an error between a result of an output of the neural network model and teacher data.

11. The model generation method according to claim 10,
wherein the neural network model is a model based on a neural network potential, and
wherein updating, by the one or more processors, the neural network model based on a method of the neural network potential.

12. The model generation method according to claim 10,
wherein the teacher data is data based on experimental data obtained in advance.

13. The model generation method according to claim 10,
wherein the teacher data is data obtained through molecular dynamics simulation.

14. The model generation method according to claim 10,
wherein obtaining, by the one or more processors, the three-dimensional structures of the plurality of molecules through molecular dynamics simulation.

15. An inferring method comprising:
obtaining, by one or more processors, three-dimensional structures of a plurality of molecules;
inputting, by the one or more processors, the three-dimensional structures of the plurality of molecules into a neural network model; and
inferring, by the one or more processors, one or more physical properties of the plurality of molecules.

16. The inferring method according to claim 15,
wherein the three-dimensional structures of the plurality of molecules are obtained through molecular dynamics simulation.

17. The inferring method according to claim 15,
wherein the three-dimensional structures of the plurality of molecules are information regarding arrangement of atoms forming the plurality of molecules.

18. An inferring method comprising:
obtaining, by one or more processors, a three-dimensional structure of one molecule through molecular dynamics simulation;
inputting, by the one or more processors, the three-dimensional structure of the one molecule into a neural network model; and
inferring, by the one or more processors, one or more physical properties of the one molecule.

19. The inferring method according to claim 18,
wherein the three-dimensional structure of the one molecule is information regarding arrangement of atoms forming the one molecule.

20. The inferring method according to claim 18,
wherein the three-dimensional structure of the one molecule further includes information regarding arrangement of atoms forming an ambient environment of the one molecule;
wherein the obtaining the three-dimensional structure of the one molecule includes obtaining both the three-dimensional structure of the one molecule and a three-dimensional structure of the ambient environment; and
wherein the inputting the three-dimensional structure of the one molecule includes inputting both the three-dimensional structure of the one molecule and the three-dimensional structure of the ambient environment into the neural network model for inferring the one or more physical properties.
